(19) United States
(12) Patent Application Publication
(10) Pub. No.: US 2022/0233603 A1
(43) Pub. Date: Jul. 28, 2022

(54) DENDRITIC CELL-BASED CANCER VACCINES AND PREPARATION METHOD THEREOF

(71) Applicant: KINKO CAPITAL CO., LTD., Tokyo (JP)

(72) Inventors: Sadatoshi SAKUMA, Yokohama-shi, Kanagawa (JP); Michiyo OSONO, Tokyo (JP); Futoshi ISHIKAWA, Tokyo (JP); Toshiki UOZUMI, Sagamihara-shi, Kanagawa (JP); Eriko OKA, Kawasaki-shi, Kanagawa (JP); Hiroki MURATA, Tokyo (JP); Hui Yu YANG, Tokyo (JP)

(21) Appl. No.: 17/614,992

(22) PCT Filed: May 27, 2019
(86) PCT No.: PCT/JP2019/020891

§ 371 Date: Nov. 29, 2021

(51) Int. Cl. A61K 35/33; A61K 39/395; A61K 35/15; C07K 14/705; A61P 35/00

(52) U.S. Cl. CPC A61K 35/33; A61K 39/39558; A61K 35/15; A61K 2039/5154; C07K 14/70525; A61P 35/00; C07K 14/70532

(57) ABSTRACT

The present invention is directed to methods for preparing a recombinant cell and a fusion cell for a dendritic cell-based cancer vaccine, wherein the recombinant cell and the fusion cell comprise DNA of a cancer cell. The present invention is also directed to the fusion cells comprising genomic DNA of a tumor cell, a method for fusing human dendritic cells and fibroblast cells, a pharmaceutical composition comprising the fusion cell, and a method of preventing cancer comprising administering to a cancer patient an effective amount of the fusion cells.

### CROSS-REFERENCE TO RELATED APPLICATIONS

This is a National Phase Application filed under 35 U.S.C. 371 as a national stage of PCT/JP2019/020891 filed May 27, 2019, the content of which is hereby incorporated by reference in its entirety.

### FIELD OF THE INVENTION

This disclosure relates to the present invention relates to methods for treating and preventing cancer by administering a vaccine comprising fusion cells formed by dendritic cells and fibroblast cells that contain genomic DNA derived from a tumor cell or a pre-cancerous cell to a cancer patient; and a fusion cell of a dendritic cell and a fibroblast cell.

### BACKGROUND OF THE INVENTION

Immunotherapeutic compositions, including vaccines, are one of the most cost-effective measures available to the healthcare industry for the prevention and treatment of disease. There remains, however, an urgent need to develop safe and effective immunotherapy strategies and adjuvants for a variety of diseases, including those caused by pathogenic agents, cancers, genetic defects and other disorders of the immune system. For the treatment of cancer and many infectious diseases, including viral diseases and diseases caused by intracellular pathogens, it is desirable to provide immunotherapy that elicits a cell-mediated (cellular) immune response, although many vaccines are directed primarily or entirely to elicitation of humoral immunity. Indeed, a disadvantage of many subunit vaccines, as well as many killed or attenuated pathogen vaccines, is that while they appear to stimulate a strong humoral immune response, they fail to elicit protective cell-mediated immunity.

Cancer is characterized primarily by an increase in the number of abnormal cells derived from a given normal tissue, invasion of adjacent tissues by these abnormal cells, and lymphatic or blood-borne spread of malignant cells to regional lymph nodes and to distant sites (metastasis). Clinical data and molecular biologic studies indicate that cancer is a multistep process that begins with minor preneoplastic changes, which may under certain conditions progress to neoplasia. Therefore, during the progression of this multistep process, pre-cancerous cells accumulated at least one genetic allele that distinguishes a pre-cancerous cell from a normal cell. Such genetic differences can result in the expression of tumor-specific antigens, over-expression of normal cellular proteins, and/or altered cellular distribution of normal and/or tumor-specific antigens. In certain instances, these alterations may result in cell-surface expression of an altered cell-surface protein or of a normal protein that is generally not transported to the cell surface.

Numerous immunotherapy studies have been reported comparing vaccine platforms that target the same antigen, in terms of their ability to induce immune cell activity and antitumor effects.

Dendritic cells, which are potent antigen presenting cells, have recently been utilized as an adjuvant for cancer immunotherapy. Gong et al. reported that inoculation of dendritic cells fused with tumor cell induced anti-tumor immunity in mice (Gong et al., 1997, Supra). Successful clinical application of fusing dendritic cell with tumor cell has also been reported (Kugler et al., 2000, Nat Med 6, 332-336). Fusion of B cells or dendritic cells with tumor cells has been previously demonstrated to elicit anti-tumor immune responses in animal models. In particular, immunization with hybrids of tumor cells and antigen presenting cells has been shown to result in protective immunity in various rodent models.

However, it is difficult and expensive to obtain and culture living tumor tissues or cells for extracting the genomic DNA of cancer cells. As of this date, there is yet an unmet need to develop a cancer vaccine for cost effective treatment of patients suffering from cancer.

### SUMMARY OF THE INVENTION

The present disclosure is directed to a method of preventing cancer comprising administering to a mammal in need of said an effective amount of fusion cells, wherein the fusion cell is formed by fusing a dendritic cell and a fibroblast cell, and wherein the fibroblast cell displays at least one antigen specific to the cancer. The fibroblast cell comprises genomic DNA of a cancer cell. In some embodiments, the genomic DNA is isolated from a cancer cell or a paraffin embedded tumor tissue. The dendritic cell and the fibroblast cell are autologous to the mammal.

The method of the present invention further comprises administrating a molecule that stimulates a humoral immune response or a cytotoxic T cell immune response to a mammal. In some embodiments, the molecule is a cytokine or interleukin.

The mammal suffers from cancer. The cancer includes, but is not limited to, renal cell carcinoma, fibrosarcoma, myxosarcoma, liposarcoma, chondrosarcoma, osteogenic sarcoma, chordoma, angiosarcoma, endotheliosarcoma, lymphangiosarcoma, lymphangioendotheliosarcoma, synovioma, mesothelioma, Ewing's tumor, leiomyosarcoma, rhabdomyosarcoma, colon carcinoma, pancreatic cancer, breast cancer, ovarian cancer, prostate cancer, squamous cell carcinoma, basal cell carcinoma, adenocarcinoma, sweat gland carcinoma, sebaceous gland carcinoma, papillary carcinoma, papillary adenocarcinomas, cystadenocarcinoma, medullary carcinoma, bronchogenic carcinoma, hepatoma, bile duct carcinoma, choriocarcinoma, seminoma, embryonal carcinoma, Wilms' tumor, cervical cancer, testicular tumor, lung carcinoma, small cell lung carcinoma, bladder carcinoma, epithelial carcinoma, glioma, astrocytoma, medulloblastoma, craniopharyngioma, ependymoma, pinealoma, hemangioblastoma, acoustic neuroma, oligodendroglioma, meningioma, melanoma, neuroblastoma, retinoblastoma, leukemias, acute lymphocytic leukemia, acute myelocytic leukemia; chronic leukemia, polycythemia vera, lymphoma, multiple myeloma, Waldenstrom's macroglobulinemia, and heavy chain disease.

The present disclosure is also directed to a method for fusing human dendritic cells and fibroblast cells, comprising subjecting a population of dendritic cells and a population of fibroblast cells to conditions that promote cell fusion, wherein the fibroblast cells comprise genomic DNA of a cancer cell, and the genomic DNA encodes at least one antigen specific to the cancer (please supplement the relative data). The fibroblast cells are autologous to the dendritic cells. In some embodiments, the cell fusion is accomplished by electrofusion.

The present disclosure is also directed to a fusion cell comprising a dendritic cell and a fibroblast cell, wherein the fusion cell comprises genomic DNA of a tumor cell, and the genomic DNA of the tumor cell encodes at least one antigen specific to the cancer.

The present disclosure also includes to a pharmaceutical composition comprising a fusion cell of the present invention and an immunologically effective amount of an adjuvant or carrier. In some embodiment, the method further comprises a molecule that stimulates an immune response selected from the group consisting of humor immune responses and/or cytotoxic T cell responses. In some embodiments, the molecule is a cytokine or interleukin.

### BRIEF DESCRIPTION OF THE DRAWINGS

FIG. 1 is a schematic diagram shows the processes for preparation of a fibroblast cell containing genomic DNA extracted from tumor samples.

FIG. 2A is a series of images shows the slide and the results of hematoxylin and eosin (H&E) staining of the tumor tissue.

FIG. 2B is a series of images shows the slide and the results of hematoxylin and eosin (H&E) staining of the tumor tissue.

FIG. 3 is a image shows cell morphology of fibroblast in primary culture.

FIG. 4 is a schematic diagram shows cell proliferation of fibroblast in primary culture at day 0-9.

FIG. 5 is a series of images show cell morphology of fibroblast in primary culture at day 0-13.

FIG. 6 is a heatmap shows mRNA expression from different samples.

### DETAILED DESCRIPTION OF THE INVENTION

The present disclosure is directed to a method of preventing cancer comprising administering to a mammal an effective amount of fusion cells, wherein the fusion cell is formed by fusing a dendritic cell and a fibroblast cell, and the fibroblast cell displays at least one antigen specific to the cancer. The fibroblast cell comprises genomic DNA of a cancer cell.

Dendritic Cells

Dendritic cells (DC) can be isolated or generated from blood or bone marrow, or secondary lymphoid organs of the subject, such as but not limited to spleen, lymph nodes, tonsils, Peyer's patch of the intestine or bone marrow, by any of the methods known in the art. In a preferred embodiment, the dendritic cells are terminally differentiated dendritic cells. In one embodiment, dendritic cells are differentiated from human blood monocytes. In certain embodiments, the dendritic cells are autologous to the subject to whom the fusion cells of the present invention are to be administered. In alternative embodiments, the dendritic cells are allogeneic to the subject to whom the fusion cells of the present invention are to be administered.

Immune cells obtained from the sources typically comprise predominantly recirculating lymphocytes and macrophages at various stages of differentiation and maturation. Dendritic cell preparations can be enriched by standard techniques (see e.g., Current Protocols in Immunology, 7.32.1-7.32.16, John Wiley and Sons, Inc., 1997). In one embodiment, for example, dendritic cells may be enriched by depletion of T cells and adherent cells, followed by density gradient centrifugation. Dendritic cells may optionally be further purified by sorting of fluorescently-labeled cells, or by using anti-CD83 mAb magnetic beads.

Alternatively, a high yield of a relatively homogenous population of dendritic cells can be obtained by treating dendritic cell progenitors present in blood samples or bone marrow with cytokines, such as granulocyte-macrophage colony stimulating factor (GM-CSF) and interleukin 4 (IL-4). Under such conditions, monocytes differentiate into dendritic cells without cell proliferation. Further treatment with an agent such as, but not limited to, TNFα stimulates terminal differentiation of dendritic cells.

Dendritic cells are obtained from blood monocytes according to standard methods (see, e.g., Sallusto et al., 1994, J. Exp. Med. 179:1109-1118). Leukocytes from healthy blood donors are collected by leukapheresis pack or buffy coat preparation using Ficoll-Paque density gradient centrifugation and plastic adherence. If mature dendritic cells are desired, the following protocol may be used to culture dendritic cells. Cells are allowed to adhere to plastic dishes for 4 hours at 37° C. Nonadherent cells are removed and adherent monocytes are cultured for 7 days in culture media containing 0.1 μg/ml granulocyte-macrophage colony stimulating factor and 0.05 μg/ml IL-4. In order to prepare dendritic cells, tumor necrosis factor-α is added on day 5 and cells are collected on day 7.

Dendritic cells express the cell surface marker CD83. In addition, such cells characteristically express high levels of MHC class II molecules, as well as cell surface markers CD1a, CD40, CD86, CD54, and CD80, but lose expression of CD14. Other cell surface markers characteristically include the T cell markers CD2 and CD5, the B cell marker CD7 and the myeloid cell markers CD13, CD32 (FcγR II), CD33, CD36, and CD63, as well as a large number of leukocyte-associated antigens

Optionally, standard techniques such as morphological observation and immunochemical staining, can be used to verify the presence of dendritic cells. For example, the purity of dendritic cells can be assessed by flow cytometry using fluorochrome-labeled antibodies directed against one or more of the characteristic cell surface markers noted above, e.g., CD83, HLA-ABC, HLA-DR, CD1a, CD40, and/or CD54. This technique can also be used to distinguish between mature and immature dendritic cells, using fluorochrome-labeled antibodies directed against CD14, which is present in immature, but not in mature, differentiated dendritic cells.

Fibroblast Cells

The fibroblast cell of the present invention can be any fibroblast cell bearing at least one allele that distinguishes the pre-cancerous cell. The fibroblast cells may be isolated from a variety of sources, such as, but not limited to, fibroblasts, macrophages, and adipocytes of the cancer patients. The fibroblast cells may also be from a primary cell culture that may be autologous, syngeneic, or allogeneic to the patient, depending on the source of the dendritic cells to be used in preparation of the fusion cells.

The source of the fibroblast cell is selected according to the cancer to be prevented. Preferably, the fibroblast cells are autologous to the patient being treated. Any fibroblast cell can be used as long as the cell comprises at least one antigen that is specific to the target cells. In one embodiment, where the dendritic cell is allogeneic to the patient, the fibroblast cell may have at least one MHC I allele that is of the same class I MHC haplotype as the mammal being treated. In another embodiment, where the dendritic cell is autologous to the patient, the fibroblast cell may be an allogeneic or autologous to the mammal being treated.

In one embodiment, the fibroblast cells of the invention are isolated from a skin tissue that is surgically removed from the mammal that will be the recipient of the fusion-cell. Prior to use, solid pre-cancerous tissue or aggregated pre-cancerous cells should be dispersed, preferably mechanically, into a single cell suspension by standard techniques. Enzymes, such as but not limited to, collagenase and DNase may also be used to disperse cancer cells. In one embodiment, the fibroblast cells of the invention are obtained from primary cell cultures, i.e., cultures of original cells obtained from the body. In one preferred embodiment, the fibroblast cells were cultured with the blood plasma from the mammal

The amount of fibroblast cells collected should be sufficient to fuse with dendritic cells to prepare enough fusion cells for the vaccines of the invention. In one embodiment, 5×10⁷ fibroblast cells are used as starting material for the formation of fusion cells. In one embodiment, approximately 1×10⁶ to 1×10⁹ fibroblast cells are used for formation of fusion cells. In another embodiment, 5×10⁷ to 2×10⁸ fibroblast cells are used. In yet another embodiment, 1×10⁷ to 1×10¹⁰ fibroblast cells are used. The use of other quantities of fibroblast cells for preparation of fusion cells are within the scope of the invention.

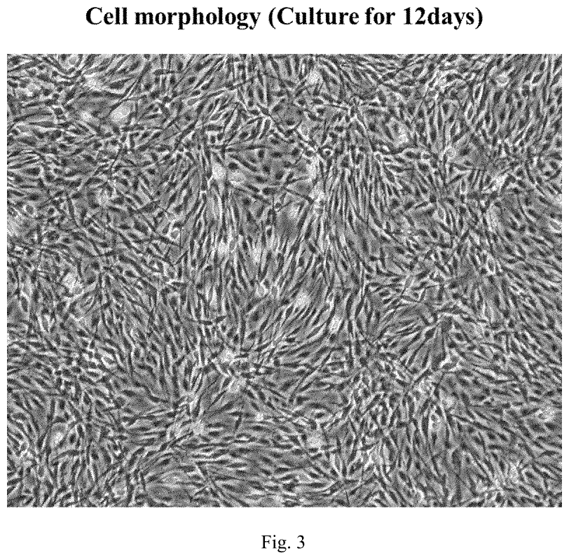

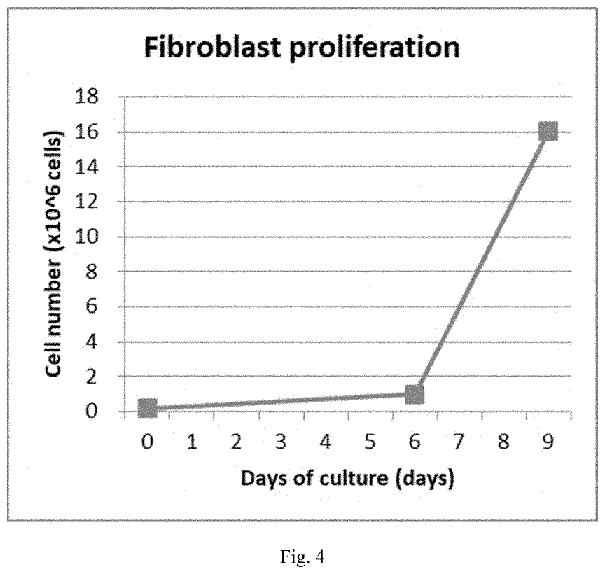

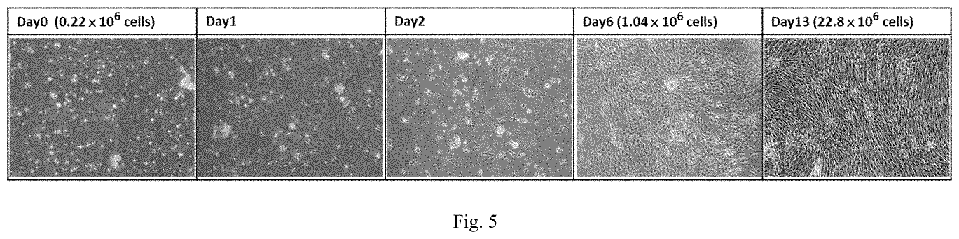

Fibroblast cells containing an antigen having the antigenicity of a cancer cell can be identified and isolated by any method known in the art. For example, fibroblast cells can be identified by morphology, enzyme assays or proliferation assays (as FIG. 3-5 shown). If the characteristics of the antigen of interest are known, fibroblast cells can also be identified or isolated by any biochemical or immunological methods known in the art. For example, fibroblast cells can be isolated by surgery, endoscopy, other biopsy techniques, affinity chromatography, and fluorescence activated cell sorting.

There is no requirement that a clonal or homogeneous or purified population of fibroblast cells be used. A mixture of cells can be used, provided that a substantial number of cells in the mixture contain the antigen being targeted. In a specific embodiment, the fibroblast cells and/or dendritic cells are purified.

Fibroblast Cells Transformed with Genomic DNA from a Tumor Cell or Paraffin Embedded Tumor Tissue

The fibroblast cells comprised genomic DNA extracted from tumor cells or pre-cancerous cells with antigen presenting cells, or paraffin embedded tumor tissues. The genomic DNA can be obtained from different sources by any method known to the skilled artisan. The genomic DNA can be transfected or microinjected into the non-dendritic cells by any method known to the skilled artisan.

The genomic DNA can be isolated or extracted from a tumor tissue or any tumor samples containing genomic DNA of a cancer cell. The tumor tissue or sample can be a living tissue/sample or a tissue specimen embedded in paraffin block. In one embodiment, the genomic DNA can be extracted from a paraffin embedded tumor tissue. In the present invention, the genomic DNA can be obtained from a sample embedded in paraffin block. Thus, the genomic DNA can be obtained more easily and cost-effectively.

In the methods of the invention, a fibroblast cell from skin tissue for the generation of fusion cells have to be capable of being transformed or microinjected with genomic DNA and have to be capable of being fused with dendritic cells. Any method known to the skilled artisan can be used to determine whether a fibroblast cell from skin tissue is suitable for the methods of the invention. In one aspect, a fibroblast cell from skin tissue is capable of being transfected or microinjected with genomic DNA. In another aspect, a fibroblast cell from skin tissue is capable of being fused with a dendritic cell.

In certain embodiments, the cell from skin tissue is derived from a species different from the species of the subject that is to be treated. Alternatively, the fibroblast cells are derived from the same species as the species of the subject that is to be treated. In certain embodiments, the fibroblast cells are heterologous to the subject that is to be treated. In other embodiments, the fibroblast cells are autologous to the subject that is to be treated. In certain embodiments, the fibroblast cells are maintained and/or propagated in cell culture.

Any method known to the skilled artisan can be used to extract genomic DNA from a cell of a tumor, cancer, or precancerous lesion. An illustrative method for isolating genomic DNA is well known in the arts. The genomic DNA can be introduced into the fibroblast cells using any method known to the skilled artisan. In certain embodiments, the genomic DNA is transfected into the fibroblast cells. In more specific embodiments, the genomic DNA is transfected into the fibroblast cells using lipofection.

The optimal amount of genomic DNA to be introduced into the fibroblast cells can be determined by standard techniques well-known to the skilled artisan. In certain embodiments, the amount of genomic DNA introduced per fibroblast cell from skin tissue corresponds to at least the equivalent of 1 genome of a tumor cell or a precancerous cell, at least the equivalent of 10⁻¹ genome of a tumor cell or a precancerous cell, at least the equivalent of 10⁻² genome of a tumor cell or a precancerous cell, at least the equivalent of 10⁻³ genome of a tumor cell or a precancerous cell, at least the equivalent of 10⁻⁴ genome of a tumor cell or a precancerous cell, at least the equivalent of 10⁻⁵ genome of a tumor cell or a precancerous cell, at least the equivalent of 10⁻⁶ genome of a tumor cell or a precancerous cell, or at least the equivalent of 10⁻⁷ genome of a tumor cell or a precancerous cell.

In certain embodiments, the genomic DNA is introduced into the fibroblast cells using microinjection. In certain embodiments, fragments of the genomic DNA are packaged into vectors for propagation of the genomic DNA. Such vectors include, but are not limited to, bacteriophages, cosmids or YACs. Any method known to the skilled artisan can be used to package and propagate the genomic DNA.

Once the genomic DNA is introduced into a fibroblast cell from skin tissue, the fibroblast cell expresses one or more of the antigens that are expressed by the tumor cell, neoplastic cell or cell of a precancerous lesion from which the genomic DNA was isolated. In certain embodiments of the invention, the fibroblast cells contain one or more molecules that display the antigenicity of the tumor or the pre-cancerous lesion. In certain embodiments, the antigen is expressed at least 2-fold, 5-fold, 10-fold, 20-fold, 50-fold or 100-fold higher levels in the tumor or the pre-cancerous lesion than in any other tissue of the subject bearing the tumor or the pre-cancerous lesion.

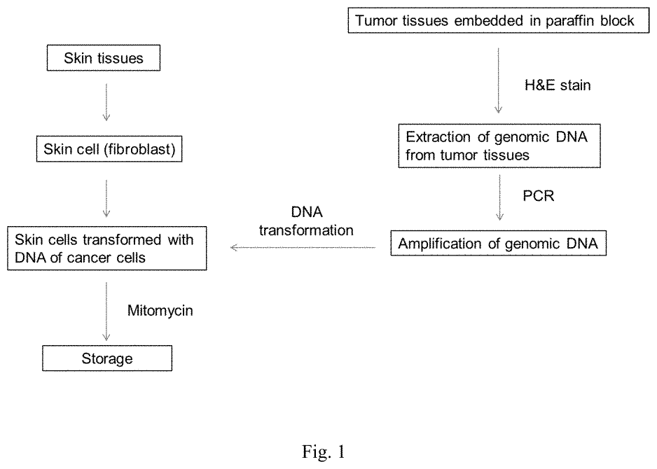

As shown in FIG. 1, a fibroblast cell from skin tissue, such as fibroblast is obtained by well-known standard techniques. A nucleotide, preferably DNA is transformed into the fibroblast cell. The DNA, such as genomic DNA of cancer cell is extracted from a tumor tissue embedded in paraffin block. Then, the fibroblast cell transformed with DNA may be treated with an antibiotic, such as mitomycin to inhibit the proliferation of the cancer cells. The fibroblast cell can be used for the subsequently fusing a dendritic cell described in detail below.

Fusion of Fibroblast Cells and Dendritic Cells

The present disclosure also includes a fusion cell of a dendritic cell and a fibroblast cell from skin tissue. The fusion cell comprises genomic DNA of a tumor cell, and the genomic DNA of the tumor cell encodes at least one antigen specific to the cancer.

The present disclosure is also directed to a method for fusing human dendritic cells and fibroblast cells, comprising subjecting a population of dendritic cells and a population of fibroblast cells to conditions that promote cell fusion.

Fibroblast cells can be fused to dendritic cells as follows. Cells are sterile-washed and fused according to any cell fusion technique in the art, provided that the fusion technique results in a mixture of fused cells suitable for injection into a mammal for prevention of cancer. Preferably, electrofusion is used. Electrofusion techniques are well known in the art (Stuhler and Walden, 1994, Cancer Immunol. Immunother. 39: 342-345; see Chang et al. (eds.), Guide to Electroporation and Electrofusion. Academic Press, San Diego, 1992).

In a preferred embodiment, the following protocol is used. In the first step, approximately 5×10⁷ pre-cancerous non-dendritic cells and 5×10⁷ dendritic cells are suspended in 0.3 M glucose and transferred into an electrofusion cuvette. The sample is dielectrophoretically aligned to form cell-cell conjugates by pulsing the cell sample at 100 V/cm for 5-10 sec. Optionally, alignment may be optimized by applying a drop of dielectrical wax onto one aspect of the electroporation cuvette to “inhomogenize” the electric field, thus directing the cells to the area of the highest field strength. In a second step, a fusion pulse is applied. Various parameters may be used for the electrofusion. For example, in one embodiment, the fusion pulse may be from a single to a triple pulse. In another embodiment, electrofusion is accomplished using from 500 to 1500V/cm, preferably, 1200V/cm at about 25 μF.

In a preferred embodiment, the following protocol is used. In the first step, non-dendritic cells are treated with 100 μg/mL of mitomycin C for 10 hours to prevent the growth of the cells. After that, the non-dendritic cells are washed with PBS, and then treated with 0.05% trypsin-EDTA. Next, the non-dendritic cells and the dendritic cells are mixed, washed and centrifuged with PBS. Then, the precipitated cells are added 0.5 mL of 50% polyethyleneglycol (PEG), warmed to 37° C., and then incubated for exactly 1 minute. Furthermore, the precipitated cells are added 7 ml of serum-free RPMI-1640 medium, and warmed to 37° C. to dilute PEG afterwards. Optionally, 8 ml of 10% FCS-containing RPMI-1640 medium is added for dilution. Lastly, PEG is removed by centrifugation, and the precipitated cells are suspended in 2% inactivated autologous plasma-added AIM-V medium supplemented with rh GM-CSF (10 ng/mL), rh IL-4 (10 ng/mL) and rh TNF-α (10 ng/mL).

In a preferred embodiment, the fibroblast cells are autologous to the patient to whom the fusion cells of the present invention are to be administered. In another preferred embodiment, the dendritic cells are autologous to the patient to whom the fusion cells of the present invention are to be administered. In an even more preferred embodiment, both the pre-cancerous non-dendritic cells and the dendritic cells are autologous to the patient to whom the fusion cells of the present invention are administered.

In another embodiment, the dendritic cell and the cell from skin tissue are fused as described above. Subsequently, the fused cells are transformed or transfected with genetic material which encodes a molecule which stimulates a CTL and/or humoral immune response. In a preferred embodiment, the genetic material is mRNA encoding IL-12. Preferred methods of transfection include electroporation or transformation or transfection in the presence of cationic polymers.

The extent of fusion cell formation within a population of fibroblast cells and dendritic cells can be determined by a number of diagnostic techniques known in the art. In one embodiment, for example, hybrids are characterized by labeling dendritic cells and fibroblast cells with red and green intracellular fluorescent dyes, respectively, and detection the emission of both colors.

Immune Cell Activating Molecules

The present invention provides a composition which comprises a fusion cell by fusing a dendritic and a fibroblast cell from skin tissue. In certain embodiments, the composition of the present invention further comprises a cytokine or other molecule which can stimulate or induce a cytotoxic T cell (CTL) response and/or a humoral response. In a preferred embodiment, the CTL stimulating molecule is IL-4, IL-12, IL-15, or IL-18.

Target Cancers

The cancers and oncogenic diseases that can be prevented, as well as the pre-cancerous lesions, which lead to the development of those cancers and oncogenic diseases, that can be prevented and treated, using the fusion cells of the present invention include, but are not limited to: human sarcomas and carcinomas, e.g., renal cell carcinoma, fibrosarcoma, myxosarcoma, liposarcoma, chondrosarcoma, osteogenic sarcoma, chordoma, angiosarcoma, endotheliosarcoma, lymphangiosarcoma, lymphangioendotheliosarcoma, synovioma, mesothelioma, Ewing's tumor, leiomyosarcoma, rhabdomyosarcoma, colon carcinoma, pancreatic cancer, breast cancer, ovarian cancer, prostate cancer, squamous cell carcinoma, basal cell carcinoma, adenocarcinoma, sweat gland carcinoma, sebaceous gland carcinoma, papillary carcinoma, papillary adenocarcinomas, cystadenocarcinoma, medullary carcinoma, bronchogenic carcinoma, hepatoma, bile duct carcinoma, choriocarcinoma, seminoma, embryonal carcinoma, Wilms' tumor, cervical cancer, testicular tumor, lung carcinoma, small cell lung carcinoma, bladder carcinoma, epithelial carcinoma, glioma, astrocytoma, medulloblastoma, craniopharyngioma, ependymoma, pinealoma, hemangioblastoma, acoustic neuroma, oligodendroglioma, meningioma, melanoma, neuroblastoma, retinoblastoma; leukemias, e.g., acute lymphocytic leukemia and acute myelocytic leukemia (myeloblastic, promyelocytic, myelomonocytic, monocytic and erythroleukemia); chronic leukemia (chronic myelocytic (granulocytic) leukemia and chronic lymphocytic leukemia); and polycythemia vera, lymphoma (Hodgkin's disease and non-Hodgkin's disease), multiple myeloma, Waldenstrom's macroglobulinemia, and heavy chain disease.

Pharmaceuticals Preparations and Methods of Administration

The present disclosure is also directed to pharmaceutical compositions containing the disclosed fusion cells.

The composition formulations of the invention comprise an effective immunizing amount of the fusion cells which are to be administered. The fusion cell of the pharmaceutical compositions of the invention can be formed by fusing an antigen-presenting cell, such as a dendritic cells or universal antigen presenting cells, and a cell from skin tissue, wherein the cell from skin tissue comprises genomic DNA extracted from a cancer cell, a cell of a precancerous lesion, or a paraffin embedded tumor tissue; cDNA or a cDNA library derived from a cancer cell, a cell of a precancerous lesion, or a paraffin embedded tumor tissue. In certain embodiments, the fusion cells of the invention express one or more antigens of the cancer to be treated or prevented.

Suitable preparations of fusion cell include injectable formulations that are, preferably, liquid solutions.

Pharmaceutical compositions can be prepared as injectables, either as liquid solutions or suspensions. The pharmaceutical composition can be administered by any suitable mode of application, for example, i.d., i.v., i.p., i.m., intranasally, orally, subcutaneously, etc. and in any suitable delivery device. In certain embodiments, the pharmaceutical composition is formulated for intravenous, subcutaneous, intradermal, or intramuscular administration. Pharmaceutical compositions suitable for other modes of administration can also be prepared, including oral and intranasal applications.

Pharmaceutical compositions can be formulated as immediate release or for sustained release formulations. Additionally, the pharmaceutical compositions can be formulated for induction of systemic, or localized mucosal, immunity through immunogen entrapment and co-administration with microparticles. Such delivery systems are readily determined by one of ordinary skill in the art.

Pharmaceutical compositions can also be formulated in a suitable dosage unit form. In some embodiments, the pharmaceutical composition contains from about 0.5 μg to about 1 mg of the tau peptide immunogen construct per kg body weight. Effective doses of the pharmaceutical compositions vary depending upon many different factors, including means of administration, target site, physiological state of the patient, whether the patient is human or an animal, other medications administered, and whether treatment is prophylactic or therapeutic. Usually, the patient is a human but nonhuman mammals including transgenic mammals can also be treated. When delivered in multiple doses, the pharmaceutical compositions may be conveniently divided into an appropriate amount per dosage unit form. The administered dosage will depend on the age, weight and general health of the subject as is well known in the therapeutic arts.

In addition, if desired, the composition preparation may also include minor amounts of auxiliary substances such as wetting or emulsifying agents, pH buffering agents, and/or compounds which enhance the effectiveness of the composition. The effectiveness of an auxiliary substance may be determined by measuring the induction of antibodies directed against a fusion cell. In some embodiments, the pharmaceutical compositions contain adjuvants or carriers such as mineral salts, including alum gel, aluminum phosphate, or water-in-oil emulsions.

The mammal to which the composition is administered is preferably a human, but can also be a non-human animal including but not limited to cows, horses, sheep, pigs, fowl (e.g., chickens), goats, cats, dogs, hamsters, mice and rats.

### Specific Embodiments

(1) A method for preparing a recombinant cell, comprising following steps:
(a) providing a fibroblast cell from a connective tissue from a mammal;
(b) extracting a genomic DNA of a cancer cell from a tumor tissue from the mammal, wherein the genomic DNA encodes at least one antigen specific to the cancer, wherein the tumor tissue from the mammal is not freshly isolated; and
(c) transforming the genomic DNA of the cancer cell to the fibroblast cell.
(2) The method according to (1), wherein the mammal is human.
(3) The method according to (1), wherein the connective tissue is a skin tissue.
(4) The method according to (1), wherein the tumor tissue from the mammal is not freshly isolated.
(5) The method according to (1), wherein the tumor tissue from the mammal embedded in a paraffin block.
(6) The method according to (3), wherein the skin tissue is maintained and washed by a skin biopsy transport and wash medium.
(7) The method according to (1), wherein the fibroblast cell is maintained and cultured in a skin biopsy culture medium, wherein the skin biopsy culture medium comprising a blood plasma from the mammal.
(8) A method for fusing dendritic cell and recombinant cell, comprising subjecting a population of dendritic cells from a mammal and a population of the recombinant cells of (1) to a condition that promote a cell fusion.
(9) The method according to (8), wherein the dendritic cells and the recombinant cells are autologous to the mammal.
(10) The method according to (8), wherein the cell fusion is accomplished by electrofusion.
(11) A recombinant cell, wherein the recombinant cell comprises a genomic DNA of a cancer cell, and the genomic DNA of the cancer cell encodes at least one antigen specific to the cancer, wherein the genomic DNA is isolated from a tumor tissue embedded in paraffin block, wherein the recombinant cell is a fibroblast from a skin tissue from a mammal, wherein the cancer cell and the fibroblast are autologous to the mammal.
(12) The recombinant cell according to (11), wherein the recombinant cell is prepared by the method of (1).
(13) A fusion cell obtained by fusing:
(a) a dendritic cell; and
(b) the recombinant cell of (1) or (11);
wherein the dendritic cell and the recombinant cell are autologous to the mammal, wherein the fusion cell comprising at least one antigen specific to a cancer, wherein the fusion cell comprising CD83, CD1a, CD40, CD86, CD54, CD80 or MHC class II.
(14) The fusion cell according to (13), wherein the fusion cell is prepared by the method of (10).
(15) A pharmaceutical composition comprising the fusion cell of (13) and an effective amount of an adjuvant or carrier.
(16) The pharmaceutical composition according to (15), further comprising a molecule that stimulates an immune response selected from the group consisting of humor immune responses, cytotoxic T cell responses, and combinations thereof.
(17) The pharmaceutical composition according to (16), wherein the molecule is a cytokine.

### Example 1

Preparation of Fibroblast Cells Containing Genomic DNA

Preparation of Skin Biopsy Transport and Wash Medium and Skin Biopsy Culture Medium

Both “skin biopsy transport and wash medium” and “skin biopsy culture medium” are prepared. The skin biopsy culture medium is personalized.

A skin biopsy transport and wash medium comprising follow ingredients: RPMI1640, fetal bovine serum, penicillin streptomycin, and gentamicin solution.

A skin biopsy culture medium comprising follow ingredients: HFDM-1(+) blood plasma from the mammal, penicillin streptomycin. The blood plasma from the mammal is autologous to the patient.

Preparation of Fibroblast Cells

A fibroblast cells were obtained from skin tissue. The skin tissue may be autologous, syngeneic, or allogeneic to the patient, depending on the source of the dendritic cells to be used in preparation of the fusion cells. The skin tissue was maintained and washed by a skin biopsy transport and wash medium. The skin tissue was homogenized by homogenizer. The fibroblast cells were maintained and cultured in a skin biopsy culture medium. The fibroblast cells are dispersed by using the Whole Skin Dissociation Kit (human) from Miltenyi biotec (Order no: 130-101-540).

The method for preparing the fibroblast cells comprising follow steps:
(a) obtaining a skin tissue;
(b) maintaining and washing the skin tissue in a skin biopsy transport and wash medium;
(c) homogenizing the skin tissue by homogenizer, and obtaining the fibroblast cells;
(d) culturing the fibroblast cells in a skin biopsy culture medium;
(e) sub-culturing the fibroblast cells in the skin biopsy culture medium;
(f) harvesting or freezing the fibroblast cells.

Primary culture protocol of dermal fibroblasts by enzyme dispersion method

1. Fibroblast Enzyme Dispersion

TABLE 1
Instruments and reagents to prepare:

| Container for collecting fibroblasts | | |
|---|---|---|
| 25 mL tube | | |
| Homogenizing tube | | |
| Sterilized tweezers | | |
| Whole Skin Dissociation Kit (Miltenyi Biotec) | | |
| Skin biopsy transport and wash medium | | |
| RPMI1640 | 200 | mL |
| Inactivated FBS | 20 | mL |
| Penicillin Streptomycin | 2 | mL |
| Gentamicin | 200 | μL |

Preparation of skin tissue collection container: 25 mL transport medium was dispensed into a skin tissue collection container and shipped to affiliated medical institution.

Inbound and received receipt of collected skin biopsy tissue: Acceptance test (sterility test with transport medium).

TABLE 2
Preparation of cell dispersion enzyme solution
(necessary amount for one tissue):

| bufferL | 435 | μL |
|---|---|---|
| EnzymeP | 12.5 | μL |
| ↓ mix | | |
| EnzymeD | 50 | μL |
| EnzymeA | 2.5 | μL |

Fibroblast Culture Operation:
(1) Prepare the enzyme solution with a container specialized for enzyme dispersion (C tube).
(2) 1 mL tissue washing medium is dispensed into a 25 mL tube. Wash the skin biopsy received from the clinic.
(3) Transfer skin biopsy to C tube, close the lid tightly. Enzymatic reaction treatment in 37° C., 5% CO₂ incubator (3 h—overnight) (In case of 3 h reaction, skin biopsy is divided into 4).

2. Sowing into Fibroblast Primary Culture T25 Flask

TABLE 3
Instruments and reagents to prepare:

100 mL tube
15 mL tube
Easy flask 25 cm²
70 μm mesh filter
1.5 mL tube
Cell counter
gentleMACS Octo Dissociator with Heaters
30 μL trypan blue for counting

TABLE 4
Preparation of primary culture medium (skin biopsy culture medium):
HFDM-1 (GF+) + 2% patient autologous plasma

| | | |
|---|---|---|
| HFDM-1 (+) NIPRO 87-160 (culture medium for culture) | 60 | mL |
| Patient autologous plasma (inactivated) | 1.2 | mL |
| Penicillin Streptmycin | 600 | μL |

Primary Culture Operation:
(1) Pour 14.5 mL of culture medium into a 15 mL tube.
(2) Take out the enzyme-dispersed container (C tube) from the incubator, from a 15 mL tube. Add 500 μL culture medium and close lid tightly.
(3) Set C tube to gentleMACS (automatic tissue disruption crusher) and start the program
(4) After completion of the program, add 2 mL culture medium. Collect in a 15 mL tube through a 70 μm mesh filter.
(5) Once again add 2 mL culture medium, centrifuge (flush). Collect in a 15 mL tube through a 70 μm mesh filter.
(6) After thoroughly stirring, 30 μL cell solution was taken, mixed with a calculation 1.5 mL tube. Load into the hemocytometer.
(7) Count the number of cells by phase contrast microscope.
(8) Centrifugation (1200 rpm, 5 min).
(9) After supernatant aspiration, suspend it in 10 mL culture medium and transfer it to a T25 flask.
(10) Cultivate in 37° C., 5% CO₂ incubator.

3. Passage from Fibroblast T25 Flask to T225 Flask.

TABLE 5
Instruments and reagents to prepare:

T 225 flask
cell counter
PBS-Buffer
0.05% TrypsinEDTA

Preparation of Fibroblast Passage:

Restore medium, PBS, trypsin EDTA to room temperature.

Passaging operation:
(1) Transfer 10 mL of culture supernatant to a T225 cm² flask.
(2) Add 5 mL PBS and then remove it, add 2 mL trypsin EDTA, digestion at room temperature for 1 minute, then remove it.
(3) Pipet 30 mL medium into T225 cm² flask.
(4) Peel off by flicking the flask, add 5 mL medium. Take a part in the 15 mL tube.
(5) Count the number of cells with an auto cell counter while keeping unstained.
(6) Transfer the cell solution to the T225 cm² flask in its entirety.
(7) Incubate at 37° C., 5% CO₂ incubator.

4. Fibroblast T225 Flasks were Frozen

TABLE 6
Instruments and reagents to prepare:

50 mL tube
cell counter
1.5 mL tube
Serum tub for freezing
PBS-Buffer
0.05% trypsin EDTA
Frozen cell preservation solution

Preparation for Fibroblast Freezing:

Restore medium, PBS, trypsin EDTA to room temperature. And issue frozen management bar code label for serum tubes.

Freezing Operation:
(1) Aspirate culture supernatant.
(2) 15 mL PBS was added and then remove, then 5 mL trypsin EDTA was added. Stay at room temperature for 1 minute.
(3) Flip 3 flask and peel off, add 11 mL medium. Transfer to a 50 mL tube.
(4) Transfer 1 mL to the specimen storage tube and store at −20° C.
(5) Count the number of cells with an auto cell counter while keeping unstained.
(6) Frozen storage cell concentration 1×10⁶ cells/mL.
(7) Centrifugation (1200 rpm, 5 min).
(8) Prepare the required number of serum tubes, paste the barcode label, enter the number of cells.
(9) After aspirating the supernatant, suspend it with 1 mL frozen cell preservation solution and calculate the solution. Suspend to the required amount.
(10) Pour into a serum tube and put it in a bicell (Cell Freezing Container).
(11) Freeze in −80° C. freezer.

FIG. 3-5 show cell morphology of fibroblast in primary culture at day 0-13, and proliferation rate of fibroblast in primary culture at day 0-9.

Isolation of Genomic DNA

A tumor tissue embedded in paraffin block was obtained from medical institution as a tumor sample. The tumor sample was stained with hematoxylin and eosin (H&E) and then determined by a doctor to confirm the antigenicity of the tumor tissue. The paraffin block was dewaxed by xylene. The tumor tissue was treated with phenol and ethanol to extract and precipitate the genomic DNA of the tumor tissue. The genomic DNA was amplified using polymerase chain reaction (PCR) and stored at −20° C.

The method for preparing the genomic DNA comprising follow steps:
(a) obtaining a tumor tissue embedded in a paraffin block;
(b) staining the paraffin block with hematoxylin and eosin (H&E) and confirming the antigenicity of the tumor tissue;
(c) dewaxing the paraffin block by xylene;
(d) extracting and precipitating the genomic DNA of the tumor tissue by phenol and ethanol;
(e) amplifying the genomic DNA by using polymerase chain reaction (PCR);
(f) storing the genomic DNA in −20° C.

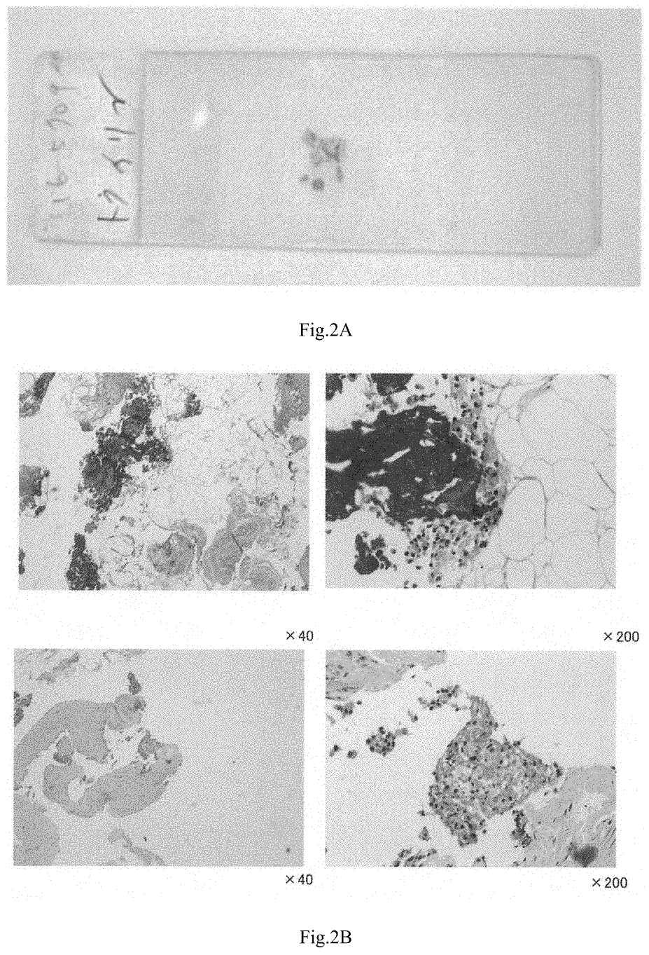

FIG. 2 shows the slide and the results of hematoxylin and eosin(H&E) staining of the tumor tissue.

The following table 7 shows the O.D.260/280 detection results of the genomic DNA sample from a cancer patient.

TABLE 7

| | | | | | | | DNA |
|---|---|---|---|---|---|---|---|
| | | | | | | | Concen- |
| Patient | Slide | | Dilution | | | | tration |
| number | number | Sample | (X) | A260 | A280 | Ratio | (μg/mL) |
| A394 | No. | PCR | 20 | 0.536 | 0.412 | 1.30 | 536.00 |
| | 16-5709 | | | | | | |

Preparation of Fibroblasts Containing Genomic DNA of Tumor Cell

A skin tissue was obtained from a cancer patient. The skin tissue was wash with skin biopsy transport and wash medium and disrupted using sonication to obtain dermal fibroblasts. The dermal fibroblasts were sub-cultured in a skin biopsy culture medium for hours. The fibroblasts were transformed with the genomic DNA obtain from tumor tissue embedded in paraffin block by lipofection. The transformed fibroblasts were cultured in transfection medium and treated with mitomycin to inhibit the proliferation of the cancer cells.

Protocol for production of cancer antigen (fibroblast+cancer genomic nucleic acid)

1. Seeding of Fibroblasts for Transfection

TABLE 8
Instruments and reagents to prepare:

6-well plate
15 mL tube
Cell counter
HFDM-1 (+)

(1) Pipette 9 mL medium into a 15 mL tube
(2) Melt the frozen cells promptly at 37° C. and transfer the cell solution to the 15 mL tube.
(3) While unstained, count the number of cells with an auto cell counter
(4) Centrifugation (1200 rpm, 5 min, RT)
(5) Discard supernatant and suspend at medium volume calculated to be 0.1×10⁶ cell/mL.
(6) Inoculate 2 mL each into 6-well plate (0.2×10⁶ cells/2 mL/well)
(7) Incubate at 37° C., 5% CO₂ incubator.

2. Transfection Operation

TABLE 9
Instruments and reagents to prepare:

1.5 mL tubes
50 mL tube
Lipofectamin 3000
Opti medium
Inactivated FBS

(1) Pipette 125 μL of opti medium into 1.5 mL tubes (L1, T1).
(2) Pipette 7.5 μL of lipofectamin 3000 into 1.5 mL tube (L1).
(3) Pour 4.0 μL of P3000 Reagent into 1.5 mL tube (T1).
(4) Add 2 μg of DNA to 1.5 mL tube (T1).
(5) Add 1.5 mL tube (L1) in the total volume to 1.5 mL tube (T1). Let stand for 10 min.
(6) Observe the cell plate for abnormalities.
(7) Discard the culture supernatant and add 2 mL 10% FBS-optiMEM.
(8) After standing, add the whole amount of 1.5 ml tube (T1) to Fibroblast, each antigen.

3. Mitomycin Treatment (Growth Inhibition of Cancer Cells)

TABLE 10
Instruments and reagents to prepare:

RPMI 1640
Inactivated FBS
Mitomycin

(1) Observe the cell plate for abnormalities.
(2) Remove the culture supernatant and add 1 mL of 10% FBS-RPMI.
(3) Add 200 μL mitomycin (500 μg/mL)(Treatment concentration 100 μg/mL).
(4) Stay at 37° C., 5% CO₂ incubator for at least 10 hr.

4. Collection and Freezing of Cancer Antigen Cells

TABLE 11
Instruments and reagents to prepare:

15 mL tube
RPMI 1640
Inactivated FBS
PBS
0.05% trypsin EDTA
Frozen cell preservation solution

(1) Dispense 7 mL 10% FBS-RPMI into a 15 mL tube.
(2) Observe the cell plate for abnormalities
(3) Collect the culture supernatant, collect the cells after washing 1 mL PBS. Add 1 mL 0.05% trypsin EDTA and leave at 37° C. for 5 minutes.
(4) Peel off the cells and collect in 15 mL tube.
(5) Centrifugation (1200 rpm, 5 min, RT).
(6) Discard the supernatant and suspend it with 2 mL PBS.
(7) Calculate the number of cells after visual counting (trypan 2-fold dilution) with a phase contrast microscope.
(8) Centrifugation (1200 rpm, 5 min, RT).
(9) Discard the supernatant, suspend it with 1000 μL frozen cell preservation solution, add it to the cryotube. Aliquot and place in a bicell (Cell Freezing Container) and freeze at −80° C.

The fibroblast cells comprise genomic DNA of a cancer cell, and the genomic DNA encodes at least one antigen specific to the cancer. To confirm the result, the following analysis method is used.

Analysis of mRNA expression by microarray of gene introduced by lipofection into fibroblast. Genomic DNA and amplified genomes by random primers transfect to fibroblasts by lipofection. The nucleic acid was introduced, and expression analysis was performed from the RNA by microarray (single color method).

TABLE 12
Samples:

Blank: Fibroblast alone
Sham: Fibroblast with only introduction treatment without using DNA
Cellline DNA: Fibroblast introducing DNA extracted from cell line
Slide PCR: Fibroblast introducing DNA amplified by PCR from DNA extracted from lung
cancer pathological biopsy
BlockDNA: Fibroblast introducing DNA extracted from lung cancer pathological specimen in a
paraffin block
BlockPCR: Fibroblast introducing DNA amplified by PCR from DNA extracted from lung
cancer pathology specimen in a paraffin block as a template

Operation Overview:
Fibroblast seeding
Transfection
Collection and freezing of Transfection sample (−80° C.)
RNA extraction
Microarray expression analysis

Microarray Analysis Overview:
Analysis contractor: Takara Bio Inc.
Name: Agilent Expression Array Analysis
Labeling method: 1 color method
CHIP type: target species human
Array name: SurePrint G3 Human GE v3 8×60 K Microarray
Design ID: 72363

“Sham” is the sample from fibroblasts with only introduction treatment without using DNA. “BlockPCR” is the sample from fibroblasts introducing DNA amplified from DNA extracted from lung cancer pathology specimen as a template. The analysis result shows gene expression difference between sham transcript (Effective judgment 0) and blockPCR transcript (Effective judgment 2). Expression difference t tests significant difference 95% or more are listed (257 results). The gene symbol and description are selectively listed below:

TABLE 13

| Gene Symbol | Gene Description |
|---|---|
| LGI1 | Homo sapiens leucine-rich, glioma inactivated 1 (LGI1), mRNA [NM_005097] |
| GHRH | Homo sapiens growth hormone releasing hormone (GHRH), transcript variant 1, |
| | mRNA [NM_021081] |
| LIPK | Homo sapiens lipase, family member K (LIPK), mRNA [NM_001080518] |
| RFPL2 | Homo sapiens ret finger protein-like 2 (RFPL2), transcript variant 1, mRNA |
| | [NM_006605] |
| lnc-EXO1-1 | LNCipedia lincRNA (lnc-EXO1-1), lincRNA [lnc-EXO1-1:1] |
| lnc-TEKT5-2 | BX089002 Soares_testis_NHT Homo sapiens cDNA clone |
| | IMAGp998K073476; IMAGE: 1377150, mRNA sequence [BX089002] |
| lnc-MACC1-1 | LNCipedia lincRNA (lnc-MACC1-1), lincRNA [lnc-MACC1-1: 5] |
| PTF1A | Homo sapiens pancreas specific transcription factor, 1a (PTF1A), mRNA |
| | [NM_178161] |
| lnc-ROS1-1 | LNCipedia lincRNA (lnc-ROS1-1), lincRNA [lnc-ROS1-1:1] |
| lnc-JPH1-3 | Homo sapiens CDNA FLJ14180 fis, clone NT2RP2003799. [AK024242] |

“BlockDNA” is the sample from fibroblasts introducing DNA extracted from lung cancer pathological specimen in a paraffin block. The analysis result shows gene expression difference between sham transcript (Effective judgment 0) and blockDNA transcript (Effective judgment 2). Expression difference t tests significant difference 95% or more are listed (410 results). The gene symbol and description are selectively listed below:

TABLE 14

| Gene Symbol | Gene Description |
|---|---|
| lnc-CPXM2-1 | LNCipedia lincRNA (lnc-CPXM2-1), lincRNA [lnc-CPXM2-1:2] |
| LINC01582 | Homo sapiens long intergenic non-protein coding RNA 1582 (LINC01582), long |
| | non-coding RNA [NR_120325] |
| LOC101927913 | PREDICTED: Homo sapiens uncharacterized LOC101927913 |
| | (LOC101927913), ncRNA [XR_241962] |
| KIAA1841 | Homo sapiens KIAA1841 (KIAA1841), transcript variant 2, mRNA |
| | [NM_032506] |
| TREM1 | Homo sapiens triggering receptor expressed on myeloid cells 1 (TREM1), |
| | transcript variant 1, mRNA [NM_018643] |
| lnc-DYRK1A-1 | LNCipedia lincRNA (lnc-DYRK1A-1), lincRNA [lnc-DYRK1A-1:1] |
| LINC00639 | Homo sapiens long intergenic non-protein coding RNA 639 (LINC00639), long |
| | non-coding RNA [NR_039982] |
| lnc-DR1-1 | LNCipedia lincRNA (lnc-DR1-1), lincRNA [lnc-DR1-1:2] |
| lnc-TNFSF15-2 | LNCipedia lincRNA (lnc-TNFSF15-2), lincRNA [lnc-TNFSF15-2:2] |
| LOC729159 | Homo sapiens UPF0607 protein ENSP00000381418-like (LOC729159), mRNA |
| | [NM_001282301] |

“SlidePCR” is the sample from fibroblast introducing DNA amplified by PCR from DNA extracted from lung cancer pathological biopsy. The analysis result shows gene expression difference between sham transcript (Effective judgment 0) and SlidePCR transcript (Effective judgment 2). Expression difference t tests significant difference 95% or more are listed (59 results). The gene symbol and description are selectively listed below:

TABLE 15

| Gene Symbol | Gene Description |
|---|---|
| lnc-NCAM2-7 | LNCipedia lincRNA (lnc-NCAM2-7), lincRNA [lnc-NCAM2-7:1] |
| UPB1 | Homo sapiens ureidopropionase, beta (UPB1), mRNA [NM_016327] |
| LOC101927637 | Homo sapiens uncharacterized LOC101927637 (LOC101927637), long non-coding |
| | RNA [NR_120451] |
| ECRP | Homo sapiens ribonuclease, RNase A family, 2 (liver, eosinophil-derived neurotoxin) |
| | pseudogene (ECRP), non-coding RNA [NR_033909] |
| lnc-TECRL-2 | LNCipedia lincRNA (lnc-TECRL-2), lincRNA [lnc-TECRL-2:1] |
| OR2AG1 | Homo sapiens olfactory receptor, family 2, subfamily AG, member 1 (gene/pseudogene) |
| | (OR2AG1), mRNA [NM_001004489] |
| CADPS | Homo sapiens Ca++-dependent secretion activator (CADPS), transcript variant 3, |
| | mRNA [NM_183393] |
| SLC38A3 | solute carrier family 38, member 3 [Source: HGNC Symbol; Acc: HGNC: 18044] |
| | [ENST00000621456] |
| OR11L1 | Homo sapiens olfactory receptor, family 11, subfamily L, member 1 (OR11L1), mRNA |
| | [NM_001001959] |
| lnc-MAP3K9-3 | LNCipedia lincRNA (lnc-MAP3K9-3), lincRNA [lnc-MAP3K9-3:6] |

“Cellline DNA” is the sample from fibroblast introducing DNA extracted from cellline. The analysis result shows gene expression difference between sham transcript (Effective judgment 0) and cellline DNA transcript (Effective judgment 2). Expression difference t tests significant difference 95% or more are listed (108 results). The gene symbol and description are selectively listed below:

TABLE 16

| Gene Symbol | Gene Description |
|---|---|
| CXCL10 | Homo sapiens chemokine (C-X-C motif) ligand 10 (CXCL10), mRNA |
| | [NM_001565] |
| CXCL11 | Homo sapiens chemokine (C-X-C motif) ligand 11 (CXCL11), transcript variant |
| | 1, mRNA [NM_005409] |
| HCP5 | Homo sapiens HLA complex P5 (non-protein coding) (HCP5), long non-coding |
| | RNA [NR_040662] |
| LOC101929371 | Homo sapiens uncharacterized LOC101929371 (LOC101929371), long non- |
| | coding RNA [NR_109861] |
| GBP1P1 | Homo sapiens guanylate binding protein 1, interferon-inducible pseudogene 1 |
| | (GBP1P1), non-coding RNA [NR_003133] |
| NRIR | Homo sapiens negative regulator of interferon response (non-protein coding) |
| | (NRIR), long non-coding RNA [NR_126359] |
| CD38 | Homo sapiens CD38 molecule (CD38), mRNA [NM_001775] |
| AIM2 | Homo sapiens absent in melanoma 2 (AIM2), mRNA [NM_004833] |
| LOC102467225 | Homo sapiens uncharacterized LOC102467225 (LOC102467225), long non- |
| | coding RNA [NR_104998] |
| GBP1P1 | guanylate binding protein 1, interferon-inducible pseudogene 1 [Source: HGNC |
| | Symbol; Acc: HGNC: 39561] [ENST00000394662] |

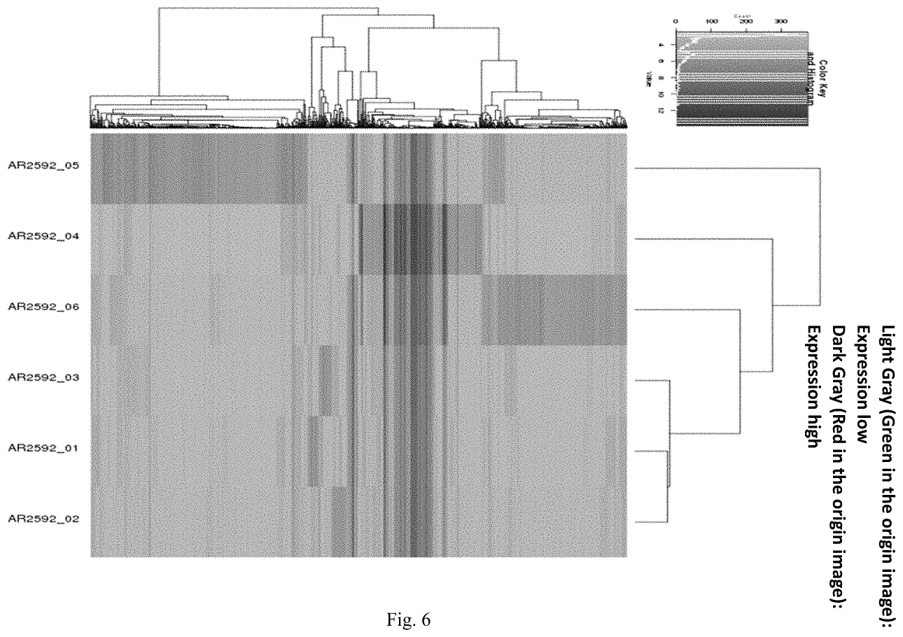

FIG. 6 shows the heatmap of mRNA expression from different samples. The phylogenetic tree created from upper 1000 genes with large variation difference.

TABLE 17
Samples:

| Sample ID | Contents |
|---|---|
| AR2592-01 | blank |
| AR2592-02 | sham |
| AR2592-03 | slide PCR |
| AR2592-04 | Cellline DNA |
| AR2592-05 | blockDNA: |
| AR2592-06 | blockPCR: |

1. A method for preparing a recombinant cell, comprising following steps:
(a) providing a fibroblast cell from a connective tissue from a mammal;
(b) extracting a genomic DNA of a cancer cell from a tumor tissue from the mammal, wherein the genomic DNA encodes at least one antigen specific to the cancer, wherein the tumor tissue from the mammal is not freshly isolated; and
(c) transforming the genomic DNA of the cancer cell to the fibroblast cell.

2. The method according to claim 1, wherein the mammal is human.

3. The method according to claim 1, wherein the connective tissue is a skin tissue.

4. The method according to claim 1, wherein the tumor tissue from the mammal is not freshly isolated.

5. The method according to claim 1, wherein the tumor tissue from the mammal embedded in a paraffin block.

6. The method according to claim 3, wherein the skin tissue is maintained and washed by a skin biopsy transport and wash medium.

7. The method according to claim 1, wherein the fibroblast cell is maintained and cultured in a skin biopsy culture medium, wherein the skin biopsy culture medium comprising a blood plasma from the mammal.

8. A method for fusing dendritic cell and recombinant cell, comprising subjecting a population of dendritic cells from a mammal and a population of the recombinant cells prepared by the method of claim 1 to a condition that promote a cell fusion.

9. The method according to claim 8, wherein the dendritic cells and the recombinant cells are autologous to the mammal.

10. The method according to claim 8, wherein the cell fusion is accomplished by electrofusion.

11. A recombinant cell, wherein the recombinant cell comprises a genomic DNA of a cancer cell, and the genomic DNA of the cancer cell encodes at least one antigen specific to the cancer, wherein the genomic DNA is isolated from a tumor tissue embedded in paraffin block, wherein the recombinant cell is a fibroblast from a skin tissue from a mammal, wherein the cancer cell and the fibroblast are autologous to the mammal.

12. The recombinant cell according to claim 11, wherein the recombinant cell is prepared by the method of claim 1.

13. A fusion cell obtained by fusing:
(a) a dendritic cell; and
(b) the recombinant cell of claim 1;
wherein the dendritic cell and the recombinant cell are autologous to the mammal, wherein the fusion cell comprising at least one antigen specific to a cancer, wherein the fusion cell comprising CD83, CD1α, CD40, CD86, CD54, CD80 or MHC class II.

14. The fusion cell according to claim 13, wherein the fusion cell is prepared by a method comprising subjecting a population of dendritic cells from a mammal and a population of the recombinant cells to a condition that promote a cell fusion, wherein the cell fusion is accomplished by electrofusion.

15. A pharmaceutical composition comprising the fusion cell of claim 13 and an effective amount of an adjuvant or carrier.

16. The pharmaceutical composition according to claim 15, further comprising a molecule that stimulates an immune response selected from the group consisting of humor immune responses, cytotoxic T cell responses, and combinations thereof.

17. The pharmaceutical composition according to claim 16, wherein the molecule is a cytokine.

18. A fusion cell obtained by fusing:
(a) a dendritic cell; and
(b) the recombinant cell of claim 11;
wherein the dendritic cell and the recombinant cell are autologous to the mammal, wherein the fusion cell comprising at least one antigen specific to a cancer, wherein the fusion cell comprising CD83, CD1α, CD40, CD86, CD54, CD80 or MHC class II.
